(19) United States
(12) Patent Application Publication
(10) Pub. No.: US 2016/0206277 A1
(43) Pub. Date: Jul. 21, 2016

(54) SYSTEMS AND METHODS FOR MONITORING HEART RATE USING ACOUSTIC SENSING

(71) Applicant: Invensense Incorporated, San Jose, CA (US)

(72) Inventors: Sameer Bidichandani, Los Gatos, CA (US); Peter Cornelius, Soquel, CA (US); Aleksey S. Khenkin, Nashua, NH (US)

(21) Appl. No.: 14/602,249

(22) Filed: Jan. 21, 2015

(51) Int. Cl. A61B 7/04; A61B 7/02; A61B 5/0205; A61B 5/00

(52) U.S. Cl. CPC A61B 7/04; A61B 5/7214; A61B 5/7278; A61B 7/026; A61B 5/0205; A61B 5/4866; A61B 5/6831; A61B 5/7246; A61B 5/6824; A61B 5/6829; A61B 5/02438

(57) ABSTRACT

Systems and methods are disclosed for heart rate measurement using a plurality of acoustic sensors in a wearable device.

### FIELD OF THE PRESENT DISCLOSURE

This disclosure generally relates to techniques for determining a user's heart rate and more particularly to heart rate measurement using a plurality of acoustic sensors in a wearable device.

### BACKGROUND

An important metric for tracking a person's health and fitness is heart rate. For example, the level of exertion associated with an activity may be accurately measured by comparing heart rate during the activity to heart rate at rest. In turn, the exertion level provides insight into the expected physiological benefits of the activity, such as quality and balance of aerobic versus anaerobic exercise and caloric consumption. Further, rates of change of heart rate between resting and active states may be used to evaluate cardiovascular health or diagnose certain diseases. Accordingly, a heart rate monitor that may be worn by a user during exercise and at rest provides valuable information.

Conventional heart rate monitors intended for personal use may be divided into two typical form factors. In one configuration, electrical sensors are arrayed on a chest strap for detecting signals associated with the user's heart beat. Although such designs offer good accuracy, they may be somewhat inconvenient or uncomfortable to wear for extended periods. Another configuration involves one or more sensors worn in a device associated with the user's hand or fingers. For example, a wristwatch type device may include a wrist sensor and a sensor pad that the user touches with a finger from the opposite hand to detect electrical signals from which the heart rate is calculated. This configuration often does not provide the level of accuracy associated with a chest strap and may also require the user to suspend the activity while obtaining the heart rate measurement.

Another hand-oriented heart rate monitor design involves an optical sensor worn adjacent the user's wrist or finger that may be used to obtain pulse oximetry signals from which heart rate is calculated. Although this design may improve accuracy, it is subject to interference from ambient light which can limit utility. Further, an illumination source may be required so that the optical sensor can measure the varying light absorption used to characterize the user's pulse, which represents a power drain. Heart rate monitors having this design may also require careful alignment of the illumination source and the optical sensor and thus may be challenging to fit properly to the user.

Correspondingly, there remains a need for a heart rate monitor to provide ongoing measurement of heart rate. Similarly, there is a need for such a monitor that may be conveniently worn during activity as well as rest. There is a further need for a heart rate monitor that reduces the amount of interaction from the user needed to obtain measurements. Still further, there is a need for a heart rate monitor design that is less subject to environmental interference and functions with reduced power requirements. This disclosure satisfies these and other needs as described in the following materials.

### SUMMARY

As will be described in detail below, this disclosure includes a method for monitoring a heart rate of a user by obtaining signals from a plurality of acoustic sensors of a wearable monitor, identifying sequential heart beats using obtained signals and calculating the heart rate based at least in part on an interval between the sequential heart beats.

This disclosure also includes a heart rate monitor having a wearable band, a plurality of acoustic sensors disposed on the wearable band and an acoustic sensing block configured to identify sequential heart beats using signals from the plurality of acoustic sensors and to calculate the heart rate based at least in part on an interval between the sequential heart beats.

### BRIEF DESCRIPTION OF THE DRAWINGS

FIG. 1 shows a user with a wearable device for monitoring heart rate with acoustic sensing according to an embodiment.

FIG. 2 is an elevational view of a device for monitoring heart rate according to an embodiment.

FIG. 3 is a schematic diagram of a device for monitoring heart rate according to an embodiment.

FIG. 4 is a flow chart of a routine for monitoring heart rate according to an embodiment.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings or chip embodiments. These and similar directional terms should not be construed to limit the scope of the disclosure in any manner.

In this specification and in the claims, it will be understood that when an element is referred to as being “connected to” or “coupled to” another element, it can be directly connected or coupled to the other element or intervening elements may be present. In contrast, when an element is referred to as being “directly connected to” or “directly coupled to” another element, there are no intervening elements present.

Some portions of the detailed descriptions which follow are presented in terms of procedures, logic blocks, processing and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments described herein may be discussed in the general context of processor-executable instructions residing on some form of non-transitory processor-readable medium, such as program blocks, executed by one or more computers or other devices. Generally, program blocks include routines, programs, objects, components, data structures, etc., that perform particular tasks or implement particular abstract data types. The functionality of the program blocks may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, blocks, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the exemplary wireless communications devices may include components other than those shown, including well-known components such as a processor, memory and the like.

The techniques described herein may be implemented in hardware, software, firmware, or any combination thereof, unless specifically described as being implemented in a specific manner. Any features described as blocks or components may also be implemented together in an integrated logic device or separately as discrete but interoperable logic devices. If implemented in software, the techniques may be realized at least in part by a non-transitory processor-readable storage medium comprising instructions that, when executed, performs one or more of the methods described above. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor. For example, a carrier wave may be employed to carry computer-readable electronic data such as those used in transmitting and receiving electronic mail or in accessing a network such as the Internet or a local area network (LAN). Of course, many modifications may be made to this configuration without departing from the scope or spirit of the claimed subject matter.

The various illustrative logical blocks, blocks, circuits and instructions described in connection with the embodiments disclosed herein may be executed by one or more processors, such as one or more motion processing units (MPUs), digital signal processors (DSPs), general purpose microprocessors, application specific integrated circuits (ASICs), application specific instruction set processors (ASIPs), field programmable gate arrays (FPGAs), or other equivalent integrated or discrete logic circuitry. The term “processor,” as used herein may refer to any of the foregoing structure or any other structure suitable for implementation of the techniques described herein. In addition, in some aspects, the functionality described herein may be provided within dedicated software blocks or hardware blocks configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an MPU and a microprocessor, a plurality of microprocessors, one or more microprocessors in conjunction with an MPU core, or any other such configuration.

Unless defined otherwise, all technical and scientific terms used herein have the same meaning as commonly understood by one having ordinary skill in the art to which the disclosure pertains.

Finally, as used in this specification and the appended claims, the singular forms “a, “an” and “the” include plural referents unless the content clearly dictates otherwise.

According to the techniques of this disclosure, a heart rate monitor may be incorporated into a device, such as a wearable device, that employs acoustic sensing to detect the sound of blood moving within a user's vessel(s). Identifying a recurring signature pattern correlated with sequential heart beats allows determination of the period, and consequently, the user's heart rate. In one aspect, the use of multiple acoustic sensors allows for the convenient application of digital signal processing techniques to selectively improve the gain of signals associated with the user's heart rate and/or reduce sound interference. Since the acoustic sensors may be incorporated into a device that is worn by the user, it may be conveniently used during activity as well as rest, requiring little or no input from the user to obtain measurements. The designs of this disclosure employ acoustic sensors, such as microphones, which may be implemented using technologies that require minimal power, enhancing their suitability for battery-dependent applications. Such acoustic sensors are also unaffected by environmental light sources, making them equally useful outdoors as well as indoors. As used herein, an acoustic sensor is any acoustic to electric transducer that converts sound carried as vibrations of a medium to an electrical signal, such as a microphone.

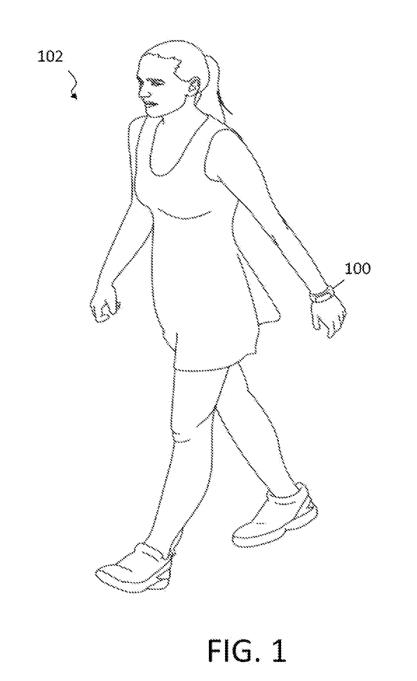

One embodiment is depicted as wrist band 100 to be worn by user 102 as shown in FIG. 1. Although the described embodiments are predominantly in the context of a wrist-worn device, other configurations are possible, such as an ankle band. Generally, a heart rate monitor according to these techniques may be configured to be worn at any location having sufficient blood volume moving during the user's heart beat to produce sufficient sound to be measured by the plurality of acoustic sensors. Further, the use of the heart rate monitor is described in the context of individual use for health or fitness, but the measured heart rate may be communicated to an external location for other monitoring purposes. For example, the devices of this disclosure may be used as an infant or patient monitor and configured to send an alert if the detected heart rate falls outside a desired range. As another example, the devices of this disclosure may be used for security to ensure that the device remains associated with a user, as a sudden failure to detect the heart beat may indicate that the device has been removed.

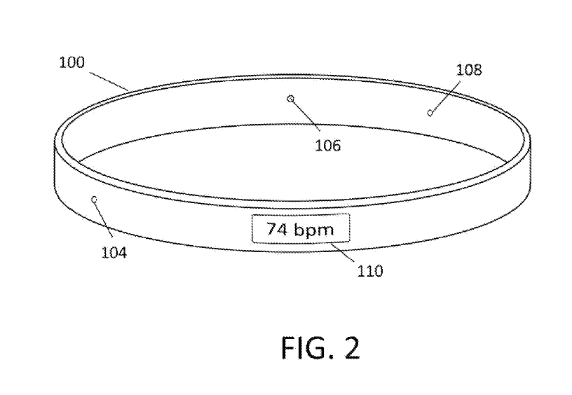

An elevational detail view of wrist band 100 is depicted in FIG. 2. As noted, a plurality of acoustic sensors may be employed, such as microphones 104, 106 and 108, to form a sensor array. The microphones may be conventional microphones configured to respond to sound waves transmitted as differences in air pressure. As desired, one or more microphones may be configured as contact microphones that respond to sound waves carried as vibrations by a medium other than air, including the user's skin. Any number of contact microphones may be used depending upon the embodiment, including all or none. In one exemplary embodiment, microphones 104 and 106 may be conventional microphones and microphone 108 may be a contact microphone. In another embodiment, all microphones 104-108 may be conventional. The acoustic sensors may also be positioned on an interior or exterior surface of wrist band to preferentially measure signals. For example, as shown in FIG. 2, microphone 104 may be positioned on the exterior to preferentially measure signals associated with ambient noise and microphones 106 and 108 may be positioned on the interior to preferentially measure signals expected to be associated with a user's heart rate.

Wrist band 100 may include display 110 to output the measured heart rate. Display 110 may also be used as a user interface to convey other information as warranted. For example, wrist band 100 may be a multi-function device, and thus may include fitness or activity tracking capabilities or other more general functions associated with a communication device (e.g., mobile or cellular phone), a watch, a personal digital assistant (PDA), a video game player and/or controller, a navigation device, a mobile internet device (MID), a personal navigation device (PND), a digital camera, a media player, a remote control, or other handheld device, or any combination of these and other similar devices. As one non-limiting example, wrist band 100 may have pedometer functions and display 110 may be used to output a variety of fitness related information, including calorie consumption derived from the measured heart rate. One or more of microphones 104-108 may serve additional purposes, such as voice pickup for communications applications.

As desired, wrist band 100 may be a self-contained device or may function in conjunction with another portable device or a non-portable device such as a desktop computer, electronic tabletop device, server computer, etc. which can communicate with wrist band 100, e.g., via network connections. The wrist band may be capable of communicating via a wired connection using any type of wire-based communication protocol (e.g., serial transmissions, parallel transmissions, packet-based data communications), wireless connection (e.g., electromagnetic radiation, infrared radiation or other wireless technology including BLUETOOTH™ (Bluetooth)), or a combination of one or more wired connections and one or more wireless connections. Therefore, although the primary embodiments discussed in this disclosure are in the context of a self-contained device, any of the functions described as being performed by wrist band 100 may be implemented in a plurality of devices as desired and depending on the relative capabilities of the respective devices. As an example, a wearable portion may incorporate the acoustic sensors that output data to another portion, such as a smart phone or tablet, which may be used to perform any or all of the other functions. As such, the term “device” may include either a self-contained device or a combination of devices acting in concert.

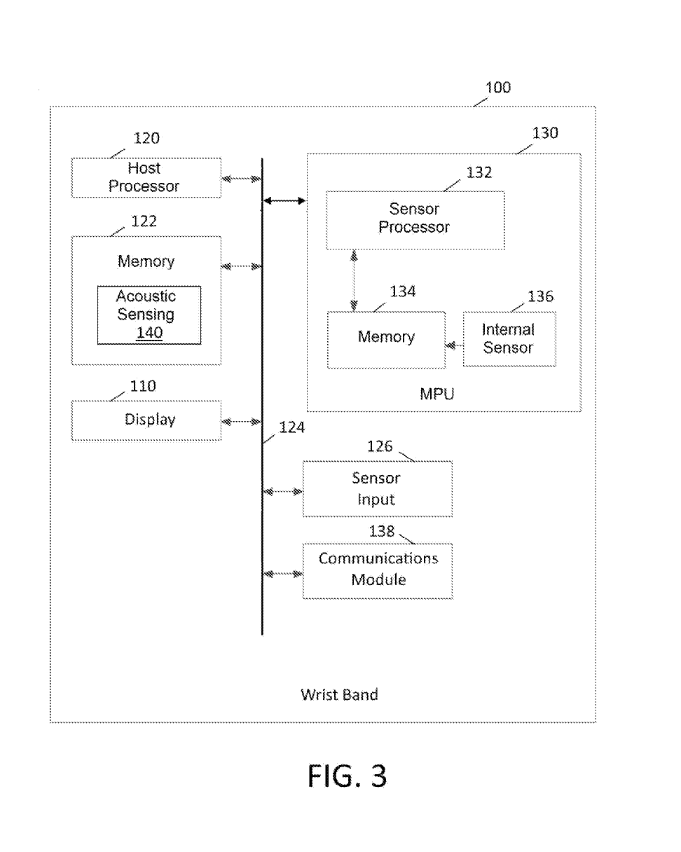

Further details of wrist band 100 are depicted schematically as high level functional blocks in FIG. 3. As shown, wrist band 100 includes host processor 120 and host memory 122 coupled by bus 124, which may be any suitable bus or interface, such as a peripheral component interconnect express (PCIe) bus, a universal serial bus (USB), a universal asynchronous receiver/transmitter (UART) serial bus, a suitable advanced microcontroller bus architecture (AMBA) interface, an Inter-Integrated Circuit (I2C) bus, a serial digital input output (SDIO) bus, or other equivalent. Host processor 120 may be one or more microprocessors, central processing units (CPUs), or other processors to run software programs or other processor-readable instructions, which may be stored in memory 122, associated with the functions of wrist band 100. Multiple layers of software can be provided in memory 122, which may be any combination of processor readable medium such as electronic, solid state memory or any other suitable storage medium, for use with the host processor 120. For example, an operating system layer can be provided for wrist band 100 to control and manage system resources in real time, enable functions of application software and other layers, and interface application programs with other software and functions of wrist band 100. Similarly, different software application programs such as menu navigation software, games, camera function control, navigation software, communications software, such as telephony or wireless local area network (WLAN) software, or any of a wide variety of other software and functional interfaces can be provided depending on the functionality of wrist band 100. As noted, multiple different applications can be provided on a single device, and in some of those embodiments, multiple applications can run simultaneously. Acoustic signals from microphones 104-108, as well as other sensors in other embodiments, are provided to sensor input 124. In some embodiments, sensor input 126 may include an analog-to-digital converter (ADC) to digitize the acoustic signals, while in other embodiments, the sensors themselves may contain ADC functionality. Display 110 may also be coupled to bus 124.

In this embodiment, wrist band 100 includes integrated motion processing unit (MPU™) 130 featuring sensor processor 132, memory 134 and internal sensor 136. Memory 134 may store algorithms, routines or other instructions for processing data output by internal sensor 136 and/or other sensors as described below using logic or controllers of sensor processor 132, as well as storing raw data and/or motion data output by internal sensor 136 or other sensors. In this embodiment, internal sensor 136 may include multiple sensors for measuring motion of wrist band 100 in space. Thus, depending on the configuration, MPU 130 measures one or more axes of rotation and/or one or more axes of acceleration of the device. In one embodiment, at least some of the motion sensors are inertial sensors, such as rotational motion sensors or linear motion sensors. For example, the rotational motion sensors may be gyroscopes to measure angular velocity along one or more orthogonal axes and the linear motion sensors may be accelerometers to measure linear acceleration along one or more orthogonal axes. In one aspect, three gyroscopes and three accelerometers may be employed, such that a sensor fusion operation performed by sensor processor 132 or other processing resources of wrist band 100 combines the motion sensor data to provide a six axis determination of motion. In one aspect, internal sensor 136 may be implemented using microelectromechanical systems (MEMS) techniques to be integrated with MPU 130 in a single package. Exemplary details regarding suitable configurations of host processor 120 and MPU 130 may be found in co-pending, commonly owned U.S. patent application Ser. No. 11/774,488, filed Jul. 6, 2007, and Ser. No. 12/106,921, filed Apr. 21, 2008, which are hereby incorporated by reference in their entirety. Suitable implementations for MPU 130 in wrist band 100 are available from InvenSense, Inc. of Sunnyvale, Calif. Similarly, any or all of microphones 104-108 may be implemented using MEMS techniques as desired.

As used herein, the term “internal sensor” refers to a sensor implemented using the MEMS techniques described above for integration with MPU 130 into a single chip. Similarly, an “external sensor” as used herein refers to a sensor carried on-board wrist band 100 that is not integrated into MPU 130. Although this embodiment is described as featuring motion sensors implemented as internal sensor 136 and microphones 104-108 implemented as external sensors, any combination of internal and/or external sensors may be used. Further, additional sensors of the same type or different may be provided either as internal or external sensors as desired. Examples of suitable sensors include accelerometers, gyroscopes, magnetometers, pressure sensors, hygrometers, barometers, microphones, photo sensors, cameras, proximity sensors and temperature sensors among others.

Wrist band 100 may also have communications module 138 to enable transfer of acoustic sensor information or other information. Communications module 138 may employ any suitable protocol, including cellular-based and wireless local area network (WLAN) technologies such as Universal Terrestrial Radio Access (UTRA), Code Division Multiple Access (CDMA) networks, Global System for Mobile Communications (GSM), the Institute of Electrical and Electronics Engineers (IEEE) 802.16 (WiMAX), Long Term Evolution (LTE), IEEE 802.11 (WiFi™) BLUETOOTH®, ZigBee®, ANT, near field communication (NFC), infrared (IR) or other technology. In one aspect, communications module 138 may be used to transmit raw or processed data from microphones 104-108 to an associated device. For example, a history of recorded heart rate measurements may be uploaded to a server. In another aspect, communications module 138 may be used to transmit signals from one or more of microphones 104-108 for other purposes, such as voice communication as noted above.

In the embodiment depicted in FIG. 3, wrist band 100 may implement functional blocks configured to perform operations associated with the techniques of this disclosure. For example, host memory 122 may include acoustic sensing block 140 receiving acoustic sensor data, such as from microphones 104-108 through sensor input 126 to identify the user's heart beat via the sound of blood rushing through the user's arteries and/or veins. Notably, acoustic sensing block 140 may employ digital sound processing techniques to isolate sounds associated with the user's heart beat. Although depicted as a functional block of processor-readable instructions stored in host memory 122 for execution by host processor 120, any desired combination of hardware, software and firmware may be employed and the functions described with respect to acoustic sensing block 140 may be performed by any combination of processing resources available to wrist band 100.

For example, acoustic sensing block 140 may combine signals from the plurality of microphones to reduce ambient noise. In one aspect, this operation may include comparing signals from one microphone with signals from one or more additional microphones. Further, different microphone designs may be employed to facilitate the comparison. In one embodiment, a first microphone may employ a unidirectional design, such as a cardioid pattern, to preferentially measure sounds from a direction within the circumference of wrist band 100 while a second microphone may employ an omnidirectional design or a directional design oriented towards locations external to the circumference to provide an indication of the ambient environment. As such, the ambient noise measured by the second microphone may be used to filter the signal of the first microphone to help isolate the signals of the first microphone expected to contain the sounds associated with the user's heart beat.

Alternatively or in addition, comparing signals from one microphone with another microphone may include employing at least one conventional microphone, such as microphones 104 and 106, and at least one contact microphone, such as microphone 108, depending on the embodiment. It may be expected that the conventional microphone(s) may provide an indication of the ambient noise field while the contact microphone(s) may provide an increased signal of the sounds associated with the user's heart beat. Any suitable combination of directional, omnidirectional, conventional and contact microphones may be employed as desired.

In another aspect, the plurality of acoustic sensors may be configured as an array to allow acoustic sensing block 140 to apply beamforming techniques as known in the art. For example, at least two microphones, such as microphones 104 and 106 may be tuned to predominantly receive signals from a desired angular direction. Notably, different weighting patterns may be applied to the signals from the microphones to control characteristics, such as width, of a main lobe representing angles from which the signals are preferentially received. Weighting patterns may also be used to control characteristics of the side lobes and a null to further refine the directions from which signals are enhanced and/or suppressed. In one embodiment, a suitable implementation of the Dolph-Chebyshev pattern may be employed. Additional microphones may be used as desired.

Acoustic sensing block 140 may also employ beam steering techniques to actively direct the main lobe of the array to a desired location. A feedback loop may be used to adjust the main lobe to increase the signal associated with the user's heart beat once identified. Generally, an array of three or more microphones may be employed when implementing beam steering.

In another aspect, acoustic sensing block 140 may use known characteristics of heart beat sounds in conjunction with a pattern matching algorithm to help identify the sound of moving blood associated with the user's heart beat. For example, each heart beat may involve regular transitions in pitch and/or amplitude. As another example, sounds associated with a user's heart beat may be expected to have frequency characteristics within a certain range. In one embodiment, a band pass filter may be used to preferentially weight signals having a frequency ranging from approximately 30 Hz to approximately 400 Hz to accommodate potential physiological minima and maxima. Alternatively, omitting a filtering stage may be employed to increase the amount of data being processed.

In yet another aspect, acoustic sensing block 140 may use information about the motion of wrist band 100 to help isolate acoustic signals associated with the user's heart beat. For example, MPU 130 may detect motion of wrist band 100 as described above. Accordingly, it may be desirable to filter out sounds that correlate with detected motion patterns, as these may be expected to be caused by movement of wrist band 100 (e.g., friction with clothing or the wrist) or the user (e.g., footfalls when a user's gait is detected) rather than the movement of blood. Alternatively or in addition, information about movement of wrist band 100 may be used in the noted pattern matching operations. For example, in activity tracking applications, MPU 130 may identify a pattern of movement associated with exercise, such as running. Based on the level of activity reflected by the movement, an expected influence on the user's heart rate may be predicted and used to suitably weight the pattern matching algorithm. Similarly, changes in a user's heart rate detected by acoustic sensing block 140 may also be used to improve the performance of MPU 130. For example, an increased heart rate may be correlated with an increased level of activity and MPU 130 may look for a more rapid pattern of motion.

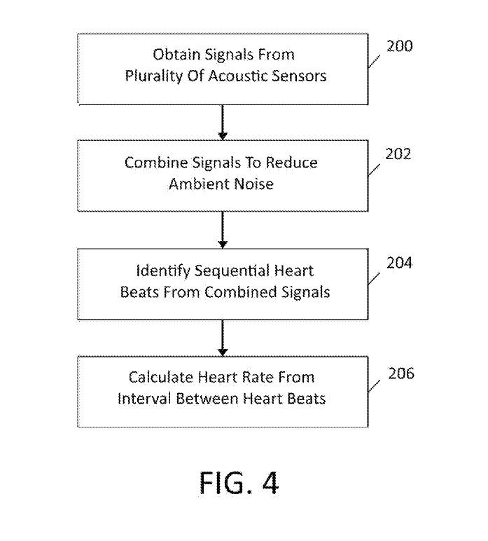

To help illustrate aspects of this disclosure, FIG. 4 depicts a flowchart showing a process for monitoring a heart rate of a user. Starting with 200, acoustic sensing block 140 may obtain signals from a plurality of acoustic sensors, such as microphones 104-108 of wrist band 100. The signals may be combined in 202 to reduce ambient noise. As noted, any combination of techniques, including the use of microphones having directional characteristics, noise cancellation, beamforming, beam steering and the like may be used. In 204, acoustic sensing block 140 identifies sequential heart beats from the combined signals. For example, acoustic sensing block 140 may employ a suitable pattern matching algorithm. Then, in 206, the user's heart rate may be calculated based at least in part on an interval between the sequential heart beats.

In the described embodiments, a chip is defined to include at least one substrate typically formed from a semiconductor material. A single chip may be formed from multiple substrates, where the substrates are mechanically bonded to preserve the functionality. A multiple chip includes at least two substrates, wherein the two substrates are electrically connected, but do not require mechanical bonding. A package provides electrical connection between the bond pads on the chip to a metal lead that can be soldered to a PCB. A package typically comprises a substrate and a cover. Integrated Circuit (IC) substrate may refer to a silicon substrate with electrical circuits, typically CMOS circuits. MEMS cap provides mechanical support for the MEMS structure. The MEMS structural layer is attached to the MEMS cap. The MEMS cap is also referred to as handle substrate or handle wafer. In the described embodiments, an electronic device incorporating a sensor may employ a motion tracking block also referred to as Motion Processing Unit (MPU) that includes at least one sensor in addition to electronic circuits. The sensor, such as a gyroscope, a compass, a magnetometer, an accelerometer, a microphone, a pressure sensor, a proximity sensor, or an ambient light sensor, among others known in the art, are contemplated. Some embodiments include accelerometer, gyroscope, and magnetometer, which each provide a measurement along three axes that are orthogonal relative to each other referred to as a 9-axis device. Other embodiments may not include all the sensors or may provide measurements along one or more axes. The sensors may be formed on a first substrate. Other embodiments may include solid-state sensors or any other type of sensors. The electronic circuits in the MPU receive measurement outputs from the one or more sensors. In some embodiments, the electronic circuits process the sensor data. The electronic circuits may be implemented on a second silicon substrate. In some embodiments, the first substrate may be vertically stacked, attached and electrically connected to the second substrate in a single semiconductor chip, while in other embodiments, the first substrate may be disposed laterally and electrically connected to the second substrate in a single semiconductor package.

In one embodiment, the first substrate is attached to the second substrate through wafer bonding, as described in commonly owned U.S. Pat. No. 7,104,129, which is incorporated herein by reference in its entirety, to simultaneously provide electrical connections and hermetically seal the MEMS devices. This fabrication technique advantageously enables technology that allows for the design and manufacture of high performance, multi-axis, inertial sensors in a very small and economical package. Integration at the wafer-level minimizes parasitic capacitances, allowing for improved signal-to-noise relative to a discrete solution. Such integration at the wafer-level also enables the incorporation of a rich feature set which minimizes the need for external amplification.

In the described embodiments, raw data refers to measurement outputs from the sensors which are not yet processed. Motion data refers to processed raw data. Processing may include applying a sensor fusion algorithm or applying any other algorithm. In the case of a sensor fusion algorithm, data from one or more sensors may be combined to provide an orientation of the device. For example, data from a 3-axis gyroscope and a 3-axis accelerometer may be combined in a 6-axis sensor fusion and data from a 3-axis gyroscope, a 3-axis accelerometer and a 3-axis magnetometer may be combined in a 9-axis sensor fusion. In the described embodiments, an MPU may include processors, memory, control logic and sensors among structures.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the present invention.

What is claimed is:

1. A method for monitoring a heart rate of a user, comprising:
obtaining signals from a plurality of acoustic sensors of a wearable monitor;
identifying sequential heart beats using obtained signals; and
calculating the heart rate based at least in part on an interval between the sequential heart beats.

2. The method of claim 1, further comprising combining the obtained signals to reduce ambient noise.

3. The method of claim 2, wherein ambient noise is identified by comparing signals from a first acoustic sensor and a second acoustic sensor of the plurality of acoustic sensors.

4. The method of claim 2, wherein at least one of the plurality of acoustic sensors comprises a contact microphone.

5. The method of claim 2, wherein combining the obtained signals comprises performing a beamforming operation.

6. The method of claim 2, wherein combining the obtained signals comprises performing a beam steering operation.

7. The method of claim 1, wherein identifying sequential heart beats comprises performing a pattern matching operation on the obtained signals from the plurality of acoustic sensors.

8. The method of claim 7, wherein performing the pattern matching operation comprises filtering a defined frequency range of the signals from the plurality of acoustic sensors.

9. The method of claim 2, further comprising detecting motion of the monitor and correlating the detected motion of the monitor with the obtained signals.

10. The method of claim 9, wherein the detected motion corresponds to a gait of the user.

11. The method of claim 9, further comprising determining a rate of calorie consumption based at least in part on the calculated heart rate and the detected motion.

12. A heart rate monitor, comprising:
a wearable band;
a plurality of acoustic sensors disposed on the wearable band; and
an acoustic sensing block,
wherein the acoustic sensing block is configured to identify sequential heart beats using signals from the plurality of acoustic sensors and to calculate the heart rate based at least in part on an interval between the sequential heart beats.

13. The heart rate monitor of claim 12, wherein the acoustic sensing block is further configured to combine the signals from the plurality of acoustic sensors to reduce ambient noise.

14. The heart rate monitor of claim 13, wherein the acoustic sensing block is configured to identify ambient noise by comparing signals from a first acoustic sensor and a second acoustic sensor of the plurality of acoustic sensors.

15. The heart rate monitor of claim 13, wherein at least one of the plurality of acoustic sensors comprises a contact microphone.

16. The heart rate monitor of claim 11, wherein the acoustic sensing block is configured to combine the signals from the plurality of acoustic sensors by performing a beamforming operation.

17. The heart rate monitor of claim 11, wherein the acoustic sensing block is configured to combine the signals from the plurality of acoustic sensors by performing a beam steering operation.

18. The heart rate monitor of claim 11, wherein the acoustic sensing block is configured to identify sequential heart beats by performing a pattern matching operation on the signals from the plurality of acoustic sensors.

19. The heart rate monitor of claim 18, wherein the acoustic sensing block is configured to perform the pattern matching operation by filtering a defined frequency range of the signals from the plurality of acoustic sensors.

20. The heart rate monitor of claim 12, further comprising a motion sensor, wherein the acoustic sensing block is configured to correlate signals from the motion sensor with signals from the plurality of acoustic sensors.

21. The heart rate monitor of claim 20, wherein the signals from the motion sensor correspond to a gait of the user.

22. The heart rate monitor of claim 20, wherein the acoustic sensing block is configured to determine a rate of calorie consumption based at least in part on the calculated heart rate and the signals from the motion sensor.

23. The heart rate monitor of claim 12, wherein the band is configured to be worn on an area of the user selected from the group consisting of a wrist and an ankle.
